(19) United States
(12) Patent Application Publication
(10) Pub. No.: US 2024/0130972 A1
(43) Pub. Date: Apr. 25, 2024

(54) FORMULATION OF A MULTI LAYERED PELLET COMPRISING MELATONIN

(71) Applicant: CLOSED LOOP MEDICINE LTD, Cambridge (GB)

(72) Inventors: Mongkol SRIWONGJANYA, Taoyuan City (TW); Lin Yu LIANG, Taoyuan City (TW); Paul GOLDSMITH, Cambridge (GB); Bruce CAMPBELL, Cambridge (GB)

(21) Appl. No.: 18/547,187

(22) PCT Filed: Feb. 24, 2022
(86) PCT No.: PCT/GB2022/050505

§ 371 Date: Aug. 20, 2023

(30) Foreign Application Priority Data
Feb. 24, 2021 (GB) 2102636.4

(51) Int. Cl. A61K 9/16; A61J 3/00; A61K 9/14; A61K 31/4045

(52) U.S. Cl. CPC A61K 9/1676; A61J 3/005; A61K 9/146; A61K 9/1682; A61K 31/4045

(57) ABSTRACT

The present Invention relates to a pellet comprising melatonin and methods of treating insomnia.

### FIELD OF THE INVENTION

The invention relates to a pharmaceutical formulation comprising melatonin.

### BACKGROUND OF THE INVENTION

Insomnia is a sleep disorder in which people have trouble sleeping. One third of the population at some point in the year have some difficulties in getting to sleep, staying asleep, or waking early. Insomnia is typically followed by daytime sleepiness, low energy, irritability, and a depressed mood. If left untreated it can lead to anxiety, depression, suicide and is related to many diseases.

Melatonin is a naturally occurring hormone released by the pineal gland that regulates the sleep-wake cycle. Previous research suggests that supplementation with melatonin may help increase total sleep time in individuals suffering from insufficient sleep or altered sleep schedules, although evidence to support this is not strong. This may be, at least in part, due to the many different types of influencing factors and causes of insomnia that mean standard treatments and dosage regimens currently available are unlikely to be effective across a range of different subjects suffering from insomnia.

Circadin is a prolonged release tablet comprising melatonin and it is a commonly used medicinal to help improve sleep quality in patients suffering with insomnia. However, there are several problems and limitations associated with Circadin, which are largely due to it being in tablet form and the potential to have a very short half-life of melatonin. For example, if the patient chews or breaks the tablet it becomes immediate release, rather than the desired prolonged release and plasma levels would quickly decline. Furthermore, tablets are more likely to be effected by differences in fed and fasted states and different types of food. Additionally, dosage amounts in prolonged release tablets cannot be readily varied according to biological and environmental factors specific to the patient and the severity of their insomnia.

### SUMMARY OF THE INVENTION

The present invention addresses the described issues with the melatonin formulations described herein. The invention provides a pellet formulation comprising melatonin that achieves a release profile that matches more closely the endogenous release levels during the night compared to immediate release tablets. The novel pellet formulation provides a loading dose for immediate sleep onset and a sustained release portion to maintain the sleeping. Furthermore, the formulation is it is cleared by wake time, as such it does not leach into the morning, unlike many other controlled release preparations. This mimics the healthy endogenous melatonin release profile and therefore potentially helps ensure sleep phenomena aren't spilling over into daytime (e.g. drowsiness or other neuroendocrine phenomena) and daytime occurrences are optimised (the body and brain primed for action). Further advantages of the pellet formulation include that it enables users to modulate the dosage by varying the number of pellets in a capsule, which in contrast to breaking prolonged release tablets, does not alter the release characteristics. It also decreases the potential to influence of food on absorption, an important consideration given it is advisable not to eat before bedtime. Additionally, the pellet formulation can be sprinkled into juice or food, to make swallowing easier, which is particularly useful for pediatrics, the elderly, patients with swallowing difficulties, such as from Parkinson's disease, Motor Neuron Disease, gastroesophageal obstructions. This results in a far more effective formulation comprising melatonin and consequently therapy than those existing currently.

Accordingly, in a first aspect of the present invention there is provided a pellet comprising:
a) a core comprising melatonin;
b) a coating layer comprising at least one polymer encompassing the core; and
c) an immediate release (IR) layer comprising melatonin encompassing the coating layer.

According to a second aspect, there is provided a capsule comprising the pellet of the invention.

According to a third aspect, there is provided a pellet or capsule of the invention for use as a medicament.

According to a fourth aspect, there is provided a method of treating insomnia comprising administering to a subject the pellet or capsule of the invention.

According to a fifth aspect, there is provided a use of the pellet or capsule of the invention in the manufacture of a medicament for the treatment of insomnia.

According to a sixth aspect, there is provided a device comprising the pellet or capsule of the invention.

According to a seventh aspect, there is provided a process for preparing a pellet, the process comprising:
a) making a core solution comprising melatonin, a coating layer solution comprising a polymer, and an IR layer solution comprising melatonin;
b) applying the core solution to a pellet and then allowing the pellet to dry, preferably until the pellet reaches a temperature of no less than 45° C.;
c) applying the coating layer solution to the pellet followed by spraying the pellet with a rinsing solution;
d) optionally curing the pellet, preferably for 2 to 6 hours, such as 3 to 5 hours; and
e) applying the IR layer solution to the pellet and then allowing the pellet to dry.

### BRIEF DESCRIPTION OF THE DRAWINGS

FIG. 1; shows the dissolution profile of Example 1 (testing conditions: 0.1N HCl, 500 ml, paddle, 50 rpm) versus the comparison tablet Circadin.

FIG. 2; shows the dissolution profile of Example 1 at 25° C. after various time periods up to 9 months.

FIG. 3; shows the dissolution profile of Example 7 (testing conditions: 55° C., 0.1N HCl, 500 ml, paddle, 50 rpm).

FIG. 4; shows the dissolution profile of Example 15 (testing conditions: 55° C., 0.1N HCl, 500 ml, paddle, 50 rpm).

FIG. 5; shows the dissolution profile of Example 16 (testing conditions: 55° C., 0.1N HCl, 500 ml, paddle, 50 rpm).

FIG. 6; shows the dissolution profile of Example 17 (testing conditions: 40° C., 0.1N HCl, 500 ml, paddle, 50 rpm).

FIG. 7; shows the dissolution profile of Example 18 (testing conditions: 55° C., 0.1N HCl, 500 ml, paddle, 50 rpm).

FIG. 8; shows schematic drawings of pellets of the invention. The skilled person will appreciate that the most inner “API” labelled ring is considered part of the “core” in accordance with the invention. The “ER” labelled ring is the “coating layer”. The outer “API” labelled ring is the “IR layer”. 8A; one embodiment wherein the pellet has three layers. 8B; a second embodiment wherein the pellet has five layers, two of which are sealing layers.

FIG. 9; shows the mean baseline melatonin plasma concentration versus time profiles following administration of Example 1 and Circadin.

### DETAILED DESCRIPTION

“Pellets” are well-known in the pharmaceutical industry. Pellets are a multi-particulate solid dosage form typically formed by the agglomeration of fine powdered excipient and Active Pharmaceutical Ingredient (API) that leads to the formation of small free flowing spherical or semi spherical particles (the pellets). The API used in the present invention is melatonin. Melatonin has a short half-life, whereas the endogenous release profile of naturally occurring melatonin in the body occurs gradually with a peak at about 4-5 hours. Therefore, it is difficult to make a formulation that matches the endogenous release profile. The typical pellet forms used in the pharmaceutical industry are single pellets with immediate release on the outside, or with the immediate release hidden behind a barrier to provide a delayed release. However, the present invention provides a pellet with a fine balance of the two forms to provide some immediate release and some slower release API.

A “capsule” is a stable shell used to enclose medicines. The two main types of capsules are hard-shelled and soft-shelled capsules. The capsules of the present invention may be hard or soft-shelled, preferably hard-shelled capsules. A preferred capsule shell of the invention is a hydroxypropyl methylcellulose (HPMC) capsule. HPMC capsule shells used herein have a lower moisture content, generally 10% w/w or less, typically 4-6% w/w and are therefore more suited for encapsulating hygroscopic substances. Preferred capsule shells of the invention have a moisture content of 10% w/w or less.

The term “treatment” includes the amelioration of the disease or condition, or a symptom or symptoms thereof. Treatment also includes the reduction in a patient's dependence on a pharmacological drug, or behaviour. “Amelioration” is an improvement, or perceived improvement, in the patient's condition, or a change in a patient's condition that makes it, or side-effects, increasingly tolerable.

The treatment of insomnia may relate to different types of insomnia. For example, the insomnia may relate to any of: sleep initiation insomnia; early morning wakening insomnia; and sleep maintenance insomnia. Additionally, the sleep initiation insomnia may be further complicated by delayed sleep phase. Patients may suffer from more than one type of insomnia. Delayed sleep phase insomnia may be defined as a patient with a delayed sleep phase component to their insomnia of greater than a 1 hour delay, or in more severe cases, greater than a 2 hour delay, between a target sleep onset time and their actual sleep onset time.

“Patient” and “subject” are used interchangeably and refer to the subject that is to receive the pellet. Preferably the subject is a human.

“weight for weight” or “weight by weight” (% w/w), refer to the proportion of a particular substance within a mixture, as measured by weight or mass.

Core

The core is at the centre of the pellet and comprises a majority of the active pharmaceutical ingredient, melatonin. The skilled person will appreciate that the core is a sphere.

In one aspect of the invention, the pellet comprises of from 0.01 to 15% w/w melatonin in the core, preferably 0.02 to 10% w/w melatonin, preferably 0.03 to 5% w/w melatonin, 0.05 to 1% w/w melatonin, more preferably 0.08 to 3% w/w melatonin, even more preferably 0.1 to 0.8% w/w melatonin, yet more preferably 0.2 to 0.6% w/w melatonin, most preferably 0.3 to 0.4% w/w melatonin.

Suitably the pellet comprises of from 0.01 mg to 20 mg of melatonin in the core, preferably 0.05 mg to 15 mg of melatonin, more preferably 0.1 mg to 10 mg of melatonin, even more preferably 0.5 mg to 8 mg of melatonin, yet more preferably 0.8 mg to 5 mg of melatonin, most preferably 1 mg to 3 mg of melatonin, such as 1 mg to 2 mg of melatonin. In one particular aspect, the pellet comprises of from 0.14 mg to 1.7 mg of melatonin in the core.

Suitably the core comprises other components. In one aspect, the core further comprises at least one excipient, preferably at least two excipients. Excipients are well-known in the pharmaceutical field and include process aids or any substances other than the active pharmaceutical ingredient that are in pharmaceutical dosage forms. Excipients help in the formulation of the pellet including helping to achieve the desired drug release profile and facilitate dosage form design.

Preferred excipients are carriers and binders. Suitably the excipient is a cellulose-based polymer, a Polyvinylpyrrolidone polymer (PVP), a starch, a sugar, or silicon dioxide, preferably a cellulose-based polymer. Suitably the cellulose-based polymer is Hydroxypropyl Cellulose, hydroxypropyl methylcellulose, microcrystalline cellulose, Hydroxyethyl cellulose, preferably microcrystalline cellulose, Hydroxypropyl Cellulose or hydroxypropyl methylcellulose, yet more preferably microcrystalline cellulose or hydroxypropyl methylcellulose, most preferably microcrystalline cellulose. In one aspect, the core comprises; (a) a sphere comprising, suitably consisting of, the excipient, and (b) the melatonin encompassing the sphere.

Suitably, the core comprises at least two excipients, selected from any of those in the preceding paragraph, preferably said at least two excipients are microcrystalline cellulose and hydroxypropyl methylcellulose.

In one aspect of the invention, the pellet comprises of from 60 to 92% w/w excipient in the core, preferably 65 to 90% w/w excipient, more preferably 70 to 88% w/w excipient, most preferably 75 to 85% w/w excipient.

Suitably the pellet comprises of from 30 mg to 650 mg of excipient in the core, preferably 100 mg to 600 mg of excipient, preferably 200 mg to 550 mg of excipient, more preferably 250 mg to 500 mg of excipient, yet more preferably 300 mg to 450 mg of excipient, most preferably 350 mg to 400 mg of excipient.

Coating Layer

The coating layer surrounds the core. The coating layer comprises a polymer and optionally a pore former. Suitably the coating layer does not comprise melatonin.

The polymer helps to achieve the desired release profile required to help treat patients with insomnia by providing a barrier around the melatonin in the core increasing the time between a patient taking the pellet and the melatonin's release. This allows for controlled, extended release of actives. The release profile can be modified by changing the amount of the polymer. The pore former is used to create a certain amount of diffusion through an insoluble coating of a tablet, pellet or particle to achieve a sustained release profile. The release profile can be modified by incorporation of a pore former and further by changing the ratio of the polymer to the pore former.

Suitably the polymer is a cellulose-based polymer, an Ethylacrylate and/or Methylmethacrylate and/or Ammonium methacrylate co-polymer, a polyvinyl alcohol, a polyacrylate, a polymethacrylate or a co-polymer thereof. Suitably the cellulose-based polymer is cellulose acetate, Cellulose Acetate Butyrate, ethylcellulose, methylcellulose, hydroxypropyl cellulose, hydroxypropylmethyl cellulose, or cellulose acetate phthalate. Preferably the polymer is cellulose acetate or ethylcellulose, more preferably cellulose acetate.

In one embodiment, the coating layer further comprises a pore former. Suitably the pore former is a poloxamer, a polyethylene-propylene glycol (PEG) copolymer, a Polyethylene Glycol, a polyalkylene glycol, a polypropylene glycol or a block co-polymer thereof, a polyglycol, an acrylic resin, a Polyvinylpyrrolidone (PVP), a cross-linked Polyvinylpyrrolidone (PVP), a polyethylene oxide, a carbomer, a diol, a polyol, a polyhydric alcohol, a poly(α-ω)alkylenediol, Triacetin, Triethyl Citrate or a Sugar alcohol, or a combination thereof. Suitable exemplary sugar alcohols include glucose, fructose, mannitol, mannose, galactose, sorbitol, pullulan and dextran. Preferably the pore former is a PEG co-polymer or a poloxamer, more preferably a poloxamer.

In one aspect of the invention, the pellet comprises of from 10 to 35% w/w polymer in the coating layer, preferably 11 to 30% w/w polymer, more preferably 12 to 25% w/w polymer, most preferably 15 to 20% w/w polymer.

In one aspect of the invention, the pellet comprises of from 0.1 to 10% w/w pore former in the coating layer, preferably 0.3 to 5% w/w pore former, more preferably 0.5 to 2% w/w pore former.

In one aspect of the invention, the pellet comprises of from 10 to 35% w/w polymer and pore former in the coating layer, preferably 11 to 30% w/w polymer and pore former, more preferably 12 to 25% w/w polymer and pore former, most preferably 15 to 20% w/w polymer and pore former.

Suitably the pellet comprises of from 10 mg to 200 mg of polymer in the coating layer, preferably 20 mg to 180 mg of polymer, more preferably 30 mg to 160 mg of polymer, yet more preferably 40 mg to 140 mg of polymer, even more preferably 50 mg to 120 mg of polymer, still more preferably 60 mg to 100 mg of polymer, most preferably 80 mg to 90 mg of polymer. In one embodiment, the pellet comprises of from 35 mg to 85 mg of polymer in the coating layer.

Suitably the pellet comprises of from 0.1 mg to 15 mg of pore former in the coating layer, preferably 0.5 mg to 10 mg of pore former, more preferably 1 mg to 8 mg of pore former, most preferably 3 mg to 6 mg of pore former.

Suitably the pellet comprises of from 10 mg to 200 mg of polymer and pore former in the coating layer, preferably 20 mg to 180 mg of polymer and pore former, more preferably 30 mg to 160 mg of polymer and pore former, yet more preferably 40 mg to 140 mg of polymer and pore former, even more preferably 50 mg to 120 mg of polymer and pore former, still more preferably 60 mg to 100 mg of polymer and pore former, most preferably 80 mg to 90 mg of polymer and pore former. In one embodiment, the pellet comprises of from 35 mg to 90 mg of polymer and pore former in the coating layer.

Immediate Release (IR) Layer

The IR layer is the outer layer of the pellet and surrounds the coating layer as shown in FIG. 8. The IR layer comprises melatonin, although typically a lower amount than the core, and helps to achieve the desired release profile required to help treat patients with insomnia. Having a small amount of melatonin in the outer layer of the pellet allows a small release of melatonin shortly after the patient has been administered the pellet helping to provide an initial quick sleep onset.

In one aspect of the invention, the pellet comprises of from 0.01 to 0.9% w/w melatonin in the IR layer, preferably 0.03 to 0.7% w/w melatonin, more preferably 0.05 to 0.5% w/w melatonin, most preferably 0.7 to 0.11% w/w melatonin.

Suitably the pellet comprises of from 0.01 to 5 mg of melatonin in the IR layer, preferably 0.05 to 3 mg of melatonin, more preferably 0.08 to 1 mg of melatonin, even more preferably 0.1 to 0.8 mg of melatonin, yet more preferably 0.3 to 0.5 mg of melatonin. In one embodiment, the pellet comprises of from 0.04 mg to 0.5 mg of melatonin in the IR layer.

Suitably the IR layer comprises other components. In one aspect, the IR layer further comprises at least one excipient. Excipients are well-known in the pharmaceutical field and include process aids or any substances other than the active pharmaceutical ingredient that are in pharmaceutical dosage forms. Excipients help in the formulation of the pellet including helping to achieve the desired drug release profile and facilitate dosage form design.

Preferred excipients are those that act as a moisture barrier minimizing the moisture level of the pellet, which in turn helps to reduce impurities that can form from hydrolysis. Suitably the excipient is a cellulose-based polymer or a Polyvinylpyrrolidone polymer (PVP), preferably a cellulose-based polymer. Preferred cellulose-based polymers are Hydroxypropyl Cellulose, hydroxypropyl methylcellulose, preferably hydroxypropyl methylcellulose.

In one aspect of the invention, the pellet comprises of from 0.0001 to 0.02% excipient in the IR layer, preferably 0.0005 to 0.015% w/w excipient, more preferably 0.001 to 0.01% w/w excipient, most preferably 0.003 to 0.008% w/w excipient.

Suitably the pellet comprises of from 0.001 mg to 1 mg of excipient in the IR layer, preferably 0.005 mg to 0.5 mg of excipient, more preferably 0.01 mg to 0.1 mg of excipient, yet more preferably 0.015 mg to 0.05 mg of excipient. In one embodiment, the pellet comprises of from 0.002 mg to 0.03 mg of excipient in the IR layer.

In one aspect, the IR layer further comprises at least one glidant. A glidant is a substance that is added to a powder to improve its flowability. Thus, the addition of the glidant further enhances the bioavailability, the desired drug release profile and facilitates the dosage form design of the pellet. The glidant is also useful to remove the statics from the pellet to increase the ease of encapsulation, talc is particularly preferential for this reason.

Exemplary glidants that may be used in the invention include talc, magnesium stearate, fumed silica (colloidal silicon dioxide), and starch, preferably talc.

In one aspect of the invention, the pellet comprises of from 0.01 to 5% w/w glidant in the IR layer, preferably 0.05 to 3% w/w glidant, more preferably 0.08 to 1% w/w glidant, even more preferably 0.1 to 0.8% w/w glidant, yet most preferably 0.3 to 0.5% w/w glidant.

Suitably the pellet comprises of from 0.01 mg to 20 mg of glidant in the IR layer, preferably 0.05 mg to 15 mg of glidant, more preferably 0.1 mg to 10 mg of glidant, yet more preferably 0.5 mg to 5 mg of glidant, most preferably 1 mg to 3 mg of glidant. In one embodiment, the pellet comprises of from 0.15 mg to 2 mg of glidant in the IR layer.

In one aspect of the invention, the total mass of the pellet is of from 40 mg to 700 mg, preferably 250 mg to 675 mg, preferably 300 mg to 650 mg, more preferably 350 mg to 600 mg, yet more preferably 400 mg to 550 mg, most preferably 450 mg to 500 mg. In one embodiment, the total mass of the pellet is of from 40 mg to 500 mg, such as 75 mg to 500 mg, for example 100 mg to 500 mg, for instance 150 mg to 500 mg.

In one aspect of the invention, the size of the pellet is of from 350 μm to 1000 μm, preferably 400 μm to 950 μm, more preferably 450 μm to 900 μm, most preferably 500 μm to 850 μm.

The pellet of the invention is stable and it can be kept in storage over time without disintegrating. Suitably the pellet can be stored stably for at least 6 months, such as at least 8 months.

Sealing Layers

In one embodiment, the pellet may further comprise one or more sealing layers. Suitably the pellet comprises a sealing layer between the core and coating layer and/or between the coating layer and the IR layer. Suitably the sealing layer does not comprise melatonin.

Sealing layers provide further encapsulation of the layers comprising melatonin to prevent the melatonin from migrating out of the layers during storage. Sealing layers are well known to the skilled person and in general, any inert material should work. Suitably the sealing layer comprises a polymer, such as a cellulose based polymer, a PVA based polymer, a PVP or a water soluble polymer, and/or the sealing layer comprises a sugar, such as lactose, and/or the sealing layer comprises a sugar alcohol, such as mannitol, preferably the sealing layer comprises a polymer. Preferably the polymer is HPMC E5 or Opadry AMB II.

In one aspect of the invention, there is provided a capsule comprising a pellet of the invention, wherein the pellet may have any of the features described throughout this disclosure. A key benefit to using capsules comprising the pellet is that it allows variable filling of the capsule with differing numbers of the pellets to achieve different dosages without having to reformulate and repeat tests in an onerous way for each dosage.

The size of the capsule may be adapted according to the number of contained pellets and thus minimise the empty air surrounding the pellets in the capsule and in turn to minimise the potential for air and moisture to impact on product stability. Accordingly, in one embodiment less than 15% of the volume inside the capsule is air, preferably less than 10%, more preferably less than 5%.

In another aspect, there is provided a device comprising a pellet or capsule of the invention, wherein the pellet or capsule may have any of the features described throughout this disclosure. Such a device allows for the dispensing of a personalised amount for a particular dosage for a specific individual.

In one embodiment of the invention, melatonin is the only active agent in the pellet, capsule or device. By only active agent it is meant that the pellet, capsule or device does not contain other components which may be used in the treatment of insomnia. Alternatively, the pellet, capsule or device does not contain other components which may be used in the treatment of any other condition or disease.

Alternatively, the pellet, capsule or device further comprises a second active agent. The second active agent may be another agent that helps improve sleep quality, such as a benzodiazepine or Z drug (for example temazepam or zopiclone), a natural product such as Valerian, lavender, passionflower or Ginkgo biloba, a sleep promoting amino acid such as 1-tryptophan or 1-theanine, magnesium, a sedative anti-histamine, doxepin, amitriptyline or mirtazapine, or orexin compounds with activity on OX1 and OX2 or both, such as suvorexant. Alternatively, the second active agent may be an active agent that treats a different disease or condition. This may be useful when a patient suffers from a disease which causes sleep interruption or from a different disease or condition as well as insomnia as it allows both active agents to be administered via the same pellet or capsule. Exemplary different disease or conditions include Parkinson's, cancer, depression, severe coughs, severe pain, or menopause. Exemplary second active agents include an anti-depressant, pain medication, cough medication, an anti-cancer agent, an anti-Parkinson's agent, oestrogen, progesterone, a corticosteroid, particularly if taken at night, such as a modified release formulation hydrocortisone microgranule.

Any suitable route may be used to administer the pellet or capsule. Preferably the route of administration is by oral, rectal, transdermal, intrathecal or parenteral (including subcutaneous, intramuscular, intravenous and intradermal) administration. Preferably the route of administration is by oral administration.

Suitably the pellet or capsule is to be administered in an effective dose, wherein the effective dose is of from 0.05 to 10 mg, preferably 0.1 to 5 mg, more preferably 0.25 to 3 mg, yet more preferably 1 to 2 mg of melatonin. Other suitable doses are 0.1, 0.15, and then increasing in increments of 0.25 mg steps to 2 mg. In specific examples, the effective dose comprises for example, 0.1, 0.2, 0.5, 0.7, 0.9, 1.0, 1.2, 1.4, 1.5, 1.7, 1.9 or 2.0 mg of melatonin. As the skilled person will understand from the disclosure, the effective dose may be achieved by administering more than one pellet or a capsule comprising more than one pellet.

As such, the invention provides a method of treating insomnia comprising administering the pellet or capsule of the invention with any of the embodiments described above to a subject in need thereof.

The invention also provides a method of assisting a subject to sleep comprising administering the pellet or capsule of the invention with any of the embodiments described above to the subject in need thereof. “To sleep” includes initiating sleep and/or maintaining sleep during the night and/or avoiding early morning wakening. In early morning wakening the subject never falls back asleep again and can be considered different from sleep maintenance in representing the body going through the normal process of full transition to the wake state, just too early. Whereas for sleep maintenance there are fluctuations in arousal during the night which are an exaggeration of what normally occurs with transitioning into prolonged wakefulness from which the patient eventually returns to sleep.

As the treatment progresses, the methods, pellets and/or capsules for use according to the various aspects of the invention described above, may be in the form of an iterative process in which the initial therapy is administered to a patient, and then as treatment progresses, the effective dose is altered to reflect how the patient is responding to the treatment to provide a modified treatment. This helps to maintain an optimal treatment of the specific patient's insomnia in their dynamic environment.

In one specific aspect of the methods, pellets and/or capsules for use, the effective dose of melatonin may be increased. This may occur, for example, if the treatment is not having the expected or desired effect on the patient i.e. the patient is still not able to get to sleep or maintain sleep in accordance with the therapy goals. Alternatively, in another aspect, the effective dose of melatonin may be decreased. This may occur, for example, if the treatment is having the expected or desired effect on the patient i.e. the patient is able to get to sleep or maintain sleep in accordance with the therapy goals and achieve targeted wake up times and total sleep time as desired. As the patient achieves this regular sleep pattern, they may be weaned off the melatonin. For example, the daily dosage amount of melatonin may be reduced each week (for example, by the dose increments described previously above) until the daily dosage amount is 0 mg, i.e. the patient no longer needs to take melatonin to treat insomnia.

Suitably the pellet or capsule is to be administered once or twice per day to be taken before bedtime, preferably once per day.

Suitably the pellet or capsule is to be administered 0 to 2 hours, preferably 30 to 60 minutes, before the preferred sleep onset time of the subject of treatment. Alternatively 1 to 2 hours before the preferred sleep onset time of the subject of treatment if there is a delayed sleep phase component to the patient's insomnia.

Typically the current controlled release melatonin formulation, Circadin, are required to be taken with food. In contrast, insomnia guidelines recommend that a patient does not eat prior to their bedtime. However, the pellet of the present invention has been formulated in such a way that taking it with food is unnecessary. Thus, in one embodiment of the invention the method, pellet or capsule for use according to the various aspects of the invention described above, may be administered to the subject, wherein the subject is not required to eat food at about the same time. Preferably the subject has not eaten food in at least the 30 minutes, preferably at least the 60 minutes, such as at least the 90 minutes, before or after the pellet or capsule is administered.

The methods and melatonin for use according to the various aspects of the invention described above may have any of the preferred features described previously in the description for other aspects of the invention.

The present invention also provides a process for preparing a pellet as described above, the process comprising:
a) making a core solution comprising melatonin, a coating layer solution comprising a polymer, and an IR layer solution comprising melatonin;
b) applying the core solution to a pellet and then allowing the pellet to dry, preferably until the pellet reaches a temperature of no less than 45° C.;
c) applying the coating layer solution to the pellet followed by spraying the pellet with a rinsing solution;
d) optionally curing the pellet, preferably for 2 to 6 hours, such as 3 to 5 hours;
e) applying the IR layer solution to the pellet and then allowing the pellet to dry.

Suitably the applying of the core solution and/or the coating layer solution and/or the IR layer solution is done by spraying.

Suitably, the method may further comprise a step of submitting the pellet for moisture testing. By “moisture testing” it means that if the moisture content of the sample pellet is Greater than 3.0%, the pellets are dried for longer, such as for at least 1 minute, such as for less than five minutes. Then a new pellet sample is collected and its moisture content is tested. This is repeated until the moisture content is no more than 3.0%. The moisture testing step may be performed after step b) and/or after step d) and/or after step e).

Suitably the method may further comprise the step f) wherein the dried pellet is passed through a multi-layer sieve shaker screen. Preferably the sieve has holes between 20 mesh (850 μm) and 35 mesh (500 μm).

Suitably the core solution may further comprise an excipient as described in the “core” section above, and/or the coating layer may further comprise a pore former as described in the “coating layer” section above, and/or the IR layer may further comprise an excipient or glidant as described in the “IR layer” section above.

Suitably the method may further comprise the step of applying a sealing layer around the core and/or the coating layer.

In another aspect, there is provided a pellet formed by the process described in the aspect of the invention above, or a capsule comprising said pellet. This process or pellet or capsule may have any of the features described earlier in the application.

### EXPERIMENTAL SECTION

### Example Pellets

Exemplary pellet formulations are shown in the tables below:

| | Example 1 | | Example 2 | | Example 3 | | Example 4 | |
|---|---|---|---|---|---|---|---|---|
| | | Weight | | Weight | | Weight | | Weight |
| | | content | | content | | content | | content |
| Components | mg | (% w/w) | mg | (% w/w) | mg | (% w/w) | mg | (% w/w) |

Core

| | | | | | | | | |
|---|---|---|---|---|---|---|---|---|
| Melatonin | 1.56 | 0.327 | 1.840 | 0.404 | 1.840 | 0.395 | 1.660 | 0.348 |
| Microcrystalline | 387.08 | 81.094 | — | — | — | — | 397.00 | 83.33 |
| Cellulose | | | | | | | | |
| Sugar sphere, NF | — | — | 397.00 | 87.18 | 397.00 | 85.21 | — | — |
| Hydroxypropyl | 0.780 | 0.163 | 1.000 | 0.220 | 1.000 | 0.215 | 0.830 | 0.174 |
| methylcellulose | | | | | | | | |
| (Hypromellose) | | | | | | | | |

Polymer coating

| | | | | | | | | |
|---|---|---|---|---|---|---|---|---|
| Cellulose Acetate | 81.220 | 17.016 | — | — | — | — | — | — |
| Ethylcellulose 7 | — | — | 48.022 | 10.546 | 57.306 | 12.300 | — | — |
| cps | | | | | | | | |
| Cellaburate | — | — | — | — | — | — | 63.90 | 13.41 |
| Poloxamer | 4.270 | 0.895 | 6.548 | 1.438 | 7.814 | 1.677 | 12.17 | 2.55 |
| (Pluronic F68) | | | | | | | | |

IR layer

| Melatonin | 0.440 | 0.092 | 0.160 | 0.035 | 0.160 | 0.034 | 0.340 | 0.071 |
|---|---|---|---|---|---|---|---|---|
| Hydroxypropyl | 0.022 | 0.005 | 0.800 | 0.176 | 0.800 | 0.172 | 0.510 | 0.107 |
| methylcellulose | | | | | | | | |
| (Hypromellose) | | | | | | | | |
| Talc | 1.950 | 0.409 | — | — | — | — | — | — |
| Total solids | 477.32 | 100 | 455.370 | 100 | 465.920 | 100 | 476.411 | 100 |
| | Example 5 | | Example 6 | | Example 7 | | Example 8 | |
| | | Weight | | Weight | | Weight | | Weight |
| | | content | | content | | content | | content |
| Components | mg | (% w/w) | mg | (% w/w) | mg | (% w/w) | mg | (% w/w) |

Core

| | | | | | | | | |
|---|---|---|---|---|---|---|---|---|
| Melatonin | 1.600 | 0.336 | 1.600 | 0.337 | 1.600 | 0.333 | 1.600 | 0.321 |
| Microcrystalline | 397.00 | 83.39 | 397.00 | 83.70 | 397.00 | 82.71 | 397.00 | 79.71 |
| Cellulose | | | | | | | | |
| Sugar sphere, | — | — | — | — | — | — | — | — |
| NF | | | | | | | | |
| Hydroxypropyl | 0.800 | 0.168 | 0.800 | 0.169 | 0.800 | 0.167 | 0.800 | 0.161 |
| methylcellulose | | | | | | | | |
| (Hypromellose) | | | | | | | | |

Polymer coating

| | | | | | | | | |
|---|---|---|---|---|---|---|---|---|
| Cellulose Acetate | — | — | 63.900 | 13.473 | 77.700 | 16.187 | — | — |
| Ethylcellulose 7 | — | — | — | — | — | — | 69.900 | 14.034 |
| cps | | | | | | | | |
| Cellaburate | 63.900 | 13.422 | — | — | — | — | — | — |
| Poloxamer | 12.171 | 2.557 | 12.171 | 2.566 | 4.089 | 0.852 | — | — |
| (Pluronic F68) | | | | | | | | |
| Hydroxypropyl | — | — | — | — | — | — | 29.957 | 6.015 |
| methylcellulose | | | | | | | | |
| (Hypromellose) | | | | | | | | |

IR layer

| Melatonin | 0.400 | 0.084 | 0.400 | | 0.084 | 0.400 | 0.083 | 0.400 | | 0.080 |
|---|---|---|---|---|---|---|---|---|---|---|
| Hydroxypropyl | 0.200 | 0.042 | 0.020 | | 0.004 | 0.020 | 0.004 | 0.020 | | 0.004 |
| methylcellulose | | | | | | | | | | |
| (Hypromellose) | | | | | | | | | | |
| Talc | — | — | — | | — | — | — | — | | — |
| Total solids | 476.071 | 100 | 474.291 | | 100 | 480.009 | 100 | 498.077 | | 100 |
| | Example 9 | | Example 10 | | Example 11 | | Example 12 | | Example 13 | |
| | | Weight | | Weight | | Weight | | Weight | | Weight |
| | | content | | content | | content | | content | | content |
| Components | mg | (% w/w) | mg | (% w/w) | mg | (% w/w) | mg | (% w/w) | mg | (% w/w) |

Core

| | | | | | | | | | | |
|---|---|---|---|---|---|---|---|---|---|---|
| Melatonin | 1.6 | 0.336 | 1.6 | 0.319 | 1.6 | 0.336 | 1.6 | 0.319 | 1.6 | 0.303 |
| Microcrystalline | 397 | 83.415 | 397 | 79.259 | 397 | 83.422 | 397 | 79.259 | 397 | 75.084 |
| Cellulose | | | | | | | | | | |
| Hydroxypropyl | 0.8 | 0.168 | 0.8 | 0.160 | 0.8 | 0.168 | 0.8 | 0.160 | 0.8 | 0.151 |
| methylcellulose | | | | | | | | | | |
| (Hypromellose) | | | | | | | | | | |

Coating layer

| | | | | | | | | | | |
|---|---|---|---|---|---|---|---|---|---|---|
| Cellaburate | 63.9 | 13.426 | 63.9 | 12.757 | 63.9 | 13.427 | 63.9 | 12.757 | 63.9 | 12.085 |
| Poloxamer | 12.171 | 2.557 | 12.171 | 2.430 | 12.171 | 2.558 | 12.171 | 2.430 | 12.171 | 2.302 |
| (Pluronic F68) | | | | | | | | | | |

Sealing layer

| | | | | | | | | | | |
|---|---|---|---|---|---|---|---|---|---|---|
| HPMC E5 | 0.04 | 0.008 | — | — | — | — | — | — | — | — |
| Opadry AMB II | — | — | 25 | 4.991 | 52.85 | 0.000 | 25 | 4.991 | 52.85 | 9.995 |
| Clear | | | | | | | | | | |

IR layer

| Melatonin | 0.4 | 0.084 | 0.4 | 0.080 | 0.4 | 0.084 | 0.4 | 0.080 | 0.4 | 0.076 |
|---|---|---|---|---|---|---|---|---|---|---|
| Hydroxypropyl | 0.02 | 0.004 | 0.02 | 0.004 | 0.02 | 0.004 | 0.02 | 0.004 | 0.02 | 0.004 |
| methylcellulose | | | | | | | | | | |
| (Hypromellose) | | | | | | | | | | |
| Total solids | 475.931 | 100 | 500.891 | 100 | 528.741 | 100 | 500.891 | 100 | 528.741 | 100 |
| | Example 14 | | Example 15 | | Example 16 | | Example 17 | | Example 18 | |
| | | Weight | | Weight | | Weight | | Weight | | Weight |
| | | content | | content | | content | | content | | content |
| Components | mg | (% w/w) | mg | (% w/w) | mg | (% w/w) | mg | (% w/w) | mg | (% w/w) |

Core

| | | | | | | | | | | |
|---|---|---|---|---|---|---|---|---|---|---|
| Melatonin | 1.6 | 0.306 | 1.6 | 0.300 | 1.6 | 0.316 | 1.6 | 0.314 | 1.6 | 0.331 |
| Microcrystalline | 397 | 75.902 | 397 | 74.404 | 397 | 78.315 | 397 | 77.983 | 397 | 82.084 |
| Cellulose | | | | | | | | | | |
| Hydroxypropyl | 0.8 | 0.153 | 0.8 | 0.150 | 0.8 | 0.158 | 0.8 | 0.157 | 0.8 | 0.165 |
| methylcellulose | | | | | | | | | | |
| (Hypromellose) | | | | | | | | | | |

Sealing layer

| | | | | | | | | | | |
|---|---|---|---|---|---|---|---|---|---|---|
| Opadry AMB II | 21 | 4.015 | — | — | — | — | — | — | — | — |
| Clear | | | | | | | | | | |
| HPMC E5 | — | — | 21 | 3.936 | 21 | 4.143 | 21 | 4.125 | 21 | 4.342 |

Coating layer

| | | | | | | | | | | |
|---|---|---|---|---|---|---|---|---|---|---|
| Cellulose | — | — | 81.8 | 15.331 | 81.8 | 16.137 | 56.55 | 11.108 | 56.55 | 11.692 |
| Acetate | | | | | | | | | | |
| Triethyl citrate | — | — | — | — | — | — | 6.283 | 1.234 | 6.283 | 1.299 |
| Cellaburate | 63.9 | 12.217 | — | — | — | — | — | — | — | — |
| Poloxamer | 12.171 | 2.430 | 12.171 | 2.302 | 12.171 | 2.327 | — | — | — | — |
| (Pluronic F68) | | | | | | | | | | |

Sealing layer

| | | | | | | | | | | |
|---|---|---|---|---|---|---|---|---|---|---|
| HPMC E5 | — | — | — | — | — | — | 26.65 | 4.995 | — | — |
| Opadry AMB II | 25 | 4.991 | 52.85 | 9.995 | 26.15 | 5.000 | — | — | — | — |
| Clear | | | | | | | | | | |

IR layer

| | | | | | | | | | | |
|---|---|---|---|---|---|---|---|---|---|---|
| Melatonin | 0.4 | 0.080 | 0.4 | 0.076 | 0.4 | 0.076 | 0.4 | 0.075 | 0.4 | 0.079 |
| Hydroxypropyl | 0.02 | 0.004 | 0.02 | 0.004 | 0.02 | 0.004 | 0.02 | 0.004 | 0.02 | 0.004 |
| methylcellulose | | | | | | | | | | |
| (Hypromellose) | | | | | | | | | | |
| Total solids | 500.891 | 100.000 | 528.741 | 100.000 | 523.041 | 100.000 | 533.575 | 100.000 | 506.925 | 100.000 |

Example pellet 1 was made by the following process. (The remaining pellets were made using the same process but with the appropriate amounts and alternative components adjusted accordingly).

Materials:

Melatonin, BP, Flamma

Microcrystalline Cellulose, NF/EP/JP/BP, Spheres 500, Weissenborn (Germany)

Hypromellose 2910/5 USP, Dow Chemical

Cellulose Acetate, USP, CA 398-10 NF/EP, Eastman

Poloxamer, NF, Kolliphor P188, BASF (USA)

Talc, EP/BP/JP/USP, Emprove, Merck

Ethanol 95.5°/c, USP, Pharmco

Acetone, NF, Pharmco-AAPER

Purified Water

Solution preparation I—Coating comprising Polymer Solution

Purified water (17.412 kg) was added to a mixer tank containing acetone (156.712 kg) and the mixture was stirred for at least 5 minutes, at 80±5 rpm. Poloxamer (555.1 g) was added to the mixer tank and stirred for at least 5 minutes, at 80±5 rpm. Then cellulose acetate (10.559 kg) was added to the mixer tank and stirred for at least 10 minutes, at 80±5 rpm. The resulting mixture was then stirred at 40±5 rpm until a clear solution was obtained.

Solution preparation II—Core Solution

A stainless steel container containing purified water (15.210 kg) was equipped with a stirrer. The stirred was turned on to achieve a vortex, and while mixing, hypromellose (101.4 g) was added to the container. Once the hypromellose was homogeneously dispersed, the speed was reduced in order to avoid foams. The mixing was continued until the solution became clear. Then, while mixing, ethanol 95.5% (5.070 kg) was added to the stainless steel container, and the mixing was continued until the solution became once again clear. While mixing, melatonin (202.8 g) was added to the stainless steel container and the resulting mixture was stirred until it became clear.

Solution preparation III—IR Solution

A stainless steel container containing purified water (15.210 kg) was equipped with a stirrer. The stirrer was turned on to achieve a vortex, then while mixing, hypromellose (2.86 g) was added to the container. Once the hypromellose was homogeneously dispersed the mixing speed was reduced in order to avoid foams. The mixing was continued until the solution became clear. Then, while mixing, ethanol (5.070 kg) was added. After the solution became clear, melatonin (57.2 g) was added to the container and the stirring was continued until the solution became clear.

Drug Layering

A fluid bed granulator was charged with microcrystalline cellulose (50.310 kg), then the microcrystalline cellulose was fluidised using air flow at 800 m³/h, at 72° C. The temperature of the cellulose pellets was monitored and when it reached no less than 34° C., the core solution (see Solution preparation II above) was sprayed in stages. Firstly, a portion of the drug layering solution (3.0 kg) was applied to the pellets (spray rate 300 g/min; inlet air temperature 74° C., air flow 900 m³/h), followed by a second portion (7.0 kg; spray rate 400 g/min; inlet air temperature 78° C.; air flow 1000 m³/h). The rest of the solution was then applied (starting with the following parameters: spray rate 500 g/min; air flow 1000 m³/h; inlet air temperature 82° C.; note: the inlet air temperature was manually adjusted within the 77-87° C. range to maintain a product temperature of 31-37° C. throughout the duration of this last stage of spraying).

The resulting pellets were then dried until they reached a temperature of no less than 45° C.

Note: at this stage of the process, a sample was submitted for moisture testing. If the moisture content is greater than 3.0%, the drying is resumed for no more than 5 minutes, then a new sample is collected and submitted for moisture testing. This is repeated until the moisture content is no more than 3.0%.

Polymer Coating

The pellets were fluidised in a fluid bed granulator (inlet air temperature 26° C.; air flow 1000 m³/h). Once the temperature of the pellets reached no more than 32° C., the polymer coating solution (see Coating solution preparation I above) was sprayed in stages. Firstly, a portion of the polymer coating solution (1.7 kg) was applied to the pellets (spray rate 350 g/min; air flow 1000 m³/h air; inlet air temperature 28° C.), followed by a second portion (4.5 kg; spray rate 450 g/min; air flow 1000 m³/h air; inlet air temperature 31° C.), followed by a third portion (9.0 kg; spray rate 550 g/min; air flow 1100 m³/h air; inlet air temperature 34° C.), followed by a fourth portion (17.5 kg; spray rate: 650 g/min; air flow 1100 m³/h air; inlet air temperature 38° C.), followed by a fifth portion (30 kg; spray rate: 750 g/min; air flow 1200 m³/h air; inlet air temperature 42° C.). Then, the rest of the polymer solution (spray rate: 750 g/min; air flow 1300 m³/h air; inlet air temperature 42° C.) was applied. Once the entire polymer coating solution was applied, the pellets were immediately sprayed with a rinsing solution (made out of acetone (9 kg) and purified water (1 kg)) for 1 to 5 minutes.

Curing

The fluidisation of the pellets was then maintained (air flow 1000 m³/h; product temperature 42-44° C.). The pellets were cured for 4 hours.

Note: at this stage of the process, another sample was submitted for moisture testing. If the moisture content is greater than 3.0%, the pellets are dried for no more than 5 minutes, then a new sample is collected and submitted for moisture testing. This is repeated until the moisture content is no more than 3.0%.

IR Coating

The pellets were then fluidised in a fluid bed granulator (air flow 1200 m³/h; inlet air temperature 72° C.). Once the product temperature reached no less than 34° C., the pellets were sprayed with the IR coating solution (see Coating solution preparation III above) in stages. Firstly, a portion of the IR solution (3.0 kg) was sprayed (spray rate 300 g/min; air flow 1300 m³/h; inlet air temperature 74° C.), followed by a second portion of the IR solution (7.0 kg; spray rate 400 g/min; air flow 1300 m³/h; inlet air temperature 78° C.), followed by the rest of the solution (spray rate 500 g/min; air flow 1300 m³/h; inlet air temperature 82° C.; note: the inlet air temperature was adjusted during this stage within the 77-87° C. range to maintain a product temperature of 31-37° C.).

The pellets were then dried and the drying process was continued until the pellets reached a product temperature of no less than 45° C.

Note: at this stage of the process, another sample was submitted for moisture testing. If the moisture content is greater than 3.0%, the pellets are dried for no more than 5 minutes, then a new sample is collected and submitted for moisture testing. This is repeated until the moisture content is no more than 3.0%.

The pellets were then discharged into a 100 L container and weighed.

The dried pellets were then passed through a multi-layer sieve shaker screen. The acceptable pellets (between 20 mesh (850 μm) and 35 mesh (500 μm)) were collected and added to a stainless steel Bin Blender.

Talc (253.5 g if the pellets were obtained in 100% yield; otherwise the talc amount must be adjusted depending on the yield of obtaining the pellets) was sieved through a 30 mesh screen. The powder was added to the Bin Blender already containing the pellets. The mixture was blended for 3 minutes at 11±1.1 rpm.

The pellets were discharged and submitted for analysis.

Dissolution and Release Profile Tests

It is difficult to make a pellet formulation that has a controlled release profile that mimics the endogenous release profile of melatonin whilst also maintaining stability over time. In order to test the stability and release profile of the exemplary pellet, the following dissolution test was performed:

Medium: 0.1N HCl

Medium volume: 500 mL

Apparatus: paddle apparatus 2 with sinker, 50 rpm

Sampling Time: 0.25, 0.5, 1, 2, 3, 4, 5, 6, 7, 8 hours. At each sampling time, the % of

melatonin released was recorded.

Sampling periods (if appropriate): 0, 2, 3, 6 and 9 months.

Results

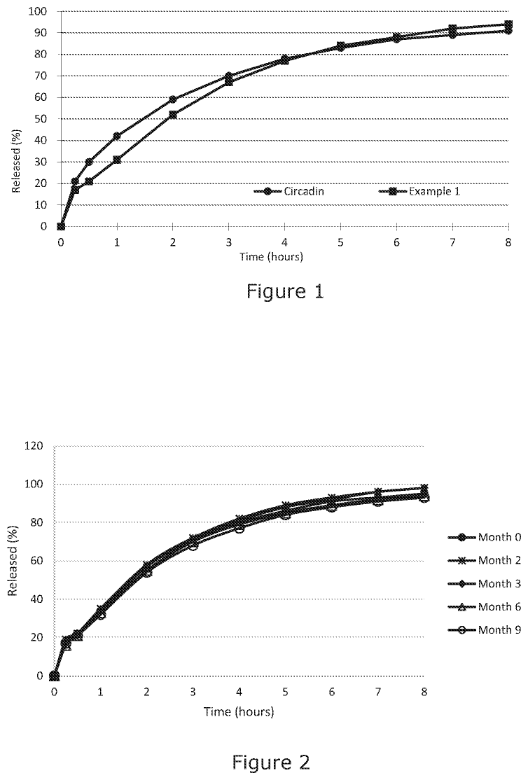

As shown in FIG. 1, Example 1 has a dissolution release profile of melatonin that matches the release profile of the commercially available prolonged release tablet, Circadin, which is accepted in the field to closely mimic the endogenous pattern of melatonin production and shows clinically efficacy on insomnia.

FIG. 2 shows that after each sampling period tested the release profile of Example 1 is substantially the same at 2, 3, 6 and 9 months as it was at 0 months showing that the pellet is stable over at least the 9 month period tested.

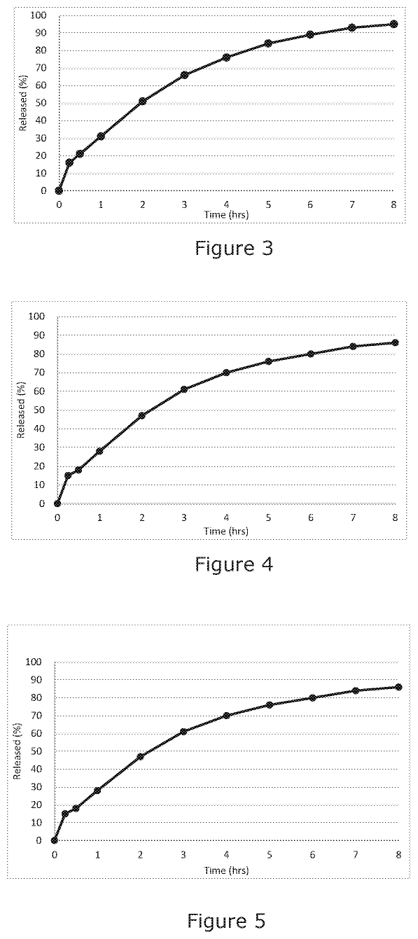

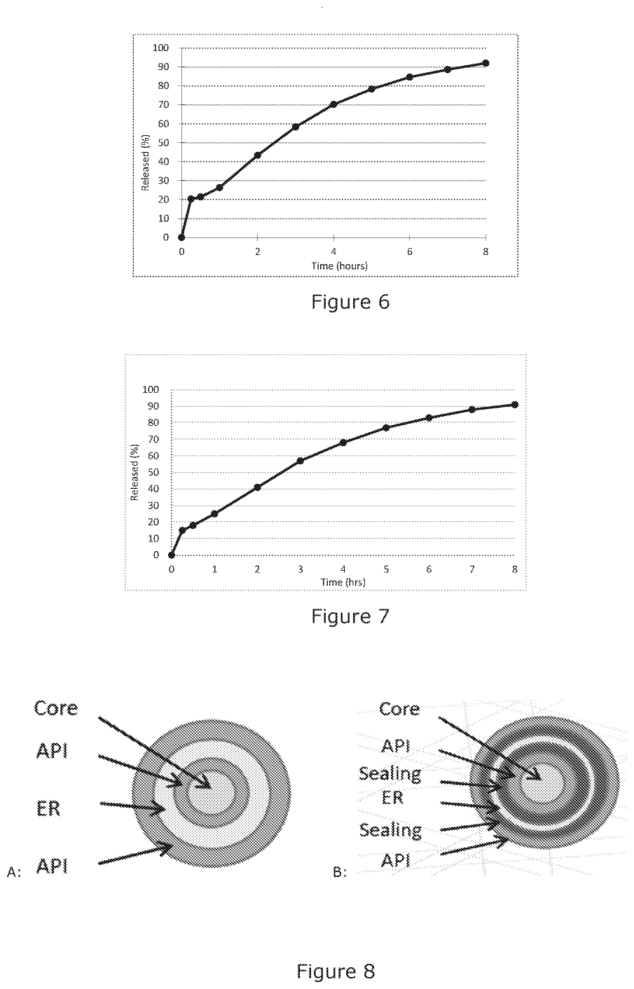

FIGS. 3 to 7 show the dissolution test results using several other example pellets. These were only performed at time 0.5 months but under stressed conditions (high temperatures) to test the stability. As can be seen in the figures, the release profiles are consistent with that of Example 1 and Circadin.

Conclusion

The pellets of the invention have high stability levels and will remain stable in storage for at least 9 months, and have a release profile that matches the endogenous release profile of melatonin. Therefore, the pellet will be useful as a medicament, particularly in the treatment of insomnia.

Bioequivalence Study of Example 1

The aims of the study were to assess the safety and tolerability of Example 1 and to demonstrate bioequivalence between Example 1 and Circadin (a 2 mg prolonged release tablet obtained from RAD Neurim Pharmaceuticals EEC SARL, which is accepted in the field to closely mimic the endogenous pattern of melatonin production and shows clinically efficacy on insomnia).

The study was performed with 46 healthy human subjects (a mixture of male and females). Melatonin plasma concentrations were measured using a validated liquid chromatography with tandem mass spectrometry (LC-MS/MS) analytical method.

Methodology

A baseline melatonin profile was collected for 24 hours prior to Period 1.

Doses of investigational medicinal product (IMP) were separated by a washout interval of 7 days.

Period 1: subjects were admitted at the clinical research facilities at least 11 hours before a baseline assessment to collect a 24-hour melatonin profile (Day −1). On completing this baseline assessment, subjects were administered Example 1 or Circadin on Day 1 and left the unit 24-hours after dosing and when all post-dose procedures were completed (Day 2). Period 2, Period 3 and Period 4: In each study period, subjects were confined from at least 11 hours before administration of Example 1 or Circadin on Day 1. Subjects left the unit 24-hours after dosing when all post-dose procedures were completed (Day 2).

In each period, confinement conditions were standardized regarding any factors that could influence melatonin pharmacokinetics, including light exposure.

According to the randomization schema, subjects were assigned to one of the two treatment sequences: 1) T-R-T-R or 2) R-T-R-T, where “T” consists of one Melatonin 2 mg prolonged release capsule (Example 1) and “R” consists of one Circadin 2 mg prolonged release tablet. The IMP was administered in the morning, orally, with 240 mL of water, 30 minutes after the start of a high-fat-high-calorie meal.

Results

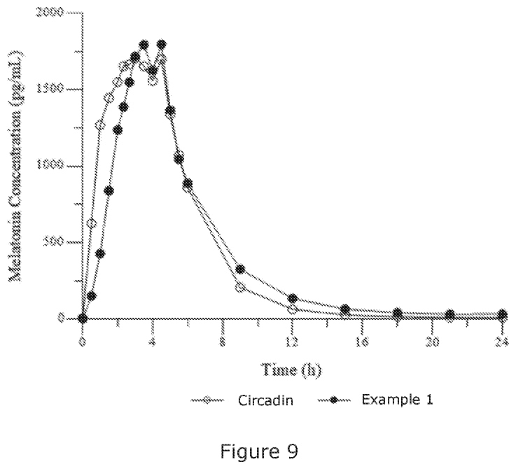

The mean baseline melatonin plasma concentration versus time profiles following administration of Example 1 and Circadin are shown in FIG. 9. The primary pharmacokinetic parameters (Cmax and AUC₀₋ₜ), the Test-to-Reference GMR and corresponding 90% CI were all within the acceptance range of 80.00 to 125.00%.

### CONCLUSIONS

Example 1 and Circadin products were similarly well tolerated with no clinically significant adverse events.

The criteria used to assess bioequivalence between Example 1 and Circadin products were fulfilled. For the primary pharmacokinetic parameters, all were within the acceptance range. Therefore, Example 1 can be considered bioequivalent to Circadin and thus to also closely mimic the endogenous pattern of melatonin.

1. A pellet comprising:
a) a core comprising melatonin;
b) a coating layer comprising at least one polymer encompassing the core; and
c) an immediate release (IR) layer comprising melatonin encompassing the coating layer.

2. The pellet of claim 1 wherein:
the core further comprises at least one excipient selected from a cellulose-based polymer, a polyvinylpyrrolidone polymer, a starch, a sugar, silicon dioxide, microcrystalline cellulose, and hydroxypropyl methylcellulose.

3. (canceled)

4. The pellet of claim 2 wherein the core comprises at least two excipients.

5. (canceled)

6. The pellet of claim 2, which comprises an amount of excipient in the core selected from 60 to 92% w/w 65 to 90% w/w, 70 to 88% w/w, and 75 to 85% w/w.

7. The pellet of claim 1, which comprises an amount of melatonin in the core selected from 0.01 to 15% w/w, 0.02 to 10% w/w, 0.03 to 5 w/w, 0.05 to 1 w/w, 0.08 to 3% w/w, 0.1 to 0.8% w/w, 0.2 to 0.6% w/w, and 0.3 to 0.4% w/w.

8. The pellet of claim 1 wherein the at least one polymer is a cellulose-based polymer, an ethylacrylate and/or methylmethacrylate and/or ammonium methacrylate co-polymer, a polyvinyl alcohol, a polyacrylate, a polymethacrylate or a co-polymer thereof.

9. The pellet of claim 8 wherein the at least one polymer is selected from cellulose acetate, cellulose acetate butyrate, ethylcellulose, methylcellulose, hydroxypropyl cellulose, hydroxypropylmethyl cellulose, and cellulose acetate phthalate.

10. The pellet of claim 1 wherein the coating layer further comprises a pore former selected from a poloxamer, a polyethylene-propylene glycol (PEG) copolymer, a Polyethylene Glycol, a polyalkylene glycol, a polypropylene glycol or a block co-polymer thereof, a polyglycol, an acrylic resin, a Polyvinylpyrrolidone (PVP), a cross-linked Polyvinylpyrrolidone (PVP), a polyethylene oxide, a carbomer, a diol, a polyol, a polyhydric alcohol, a poly(α-ω)alkylenediol, Triacetin, Triethyl Citrate or a Sugar alcohol, or combinations thereof.

11. (canceled)

12. The pellet of claim 1 wherein the pellet comprises an amount of polymer in the coating layer selected from 10 to 35% w/w, 11 to 30% w/w, 12 to 25% w/w, and 15 to 20% w/w.

13. The pellet of claim 10 wherein the pellet comprises an amount of pore former in the coating layer selected from 0.1 to 10% w/w, 0.3 to 5 w/w, and 0.5 to 2% w/w.

14. The pellet of claim 1 wherein the IR layer further comprises at least one excipient selected from a cellulose-based polymer or a polyvinylpyrrolidone polymer, hydroxypropyl cellulose, and hydroxypropyl methylcellulose.

15. (canceled)

16. The pellet of claim 1 wherein the pellet comprises an amount of excipient in the IR layer selected from 0.0001 to 0.02% w/w, 0.0005 to 0.015% w/w, 0.001 to 0.01% w/w, and 0.003 to 0.008% w/w.

17. (canceled)

18. The pellet of claim 1 wherein the pellet comprises an amount of melatonin in the IR layer selected from 0.01 to 0.9% w/w, 0.03 to 0.7% w/w, 0.05 to 0.5% w/w, and 0.7 to 0.11% w/w.

19. The pellet of claim 1 wherein the coating layer does not comprise melatonin.

20. The pellet of claim 1 wherein the size of the pellet is selected from 350 μm to 1000 μm, 400 μm to 950 μm, 450 μm to 900 μm, and 500 μm to 850 μm.

21. (canceled)

22. The pellet of claim 1 wherein the melatonin is the only active agent in the pellet.

23-25. (canceled)

26. The pellet of claim 1 which is formulated to deliver an effective dose of melatonin selected from 0.05 to 10 mg, 0.1 to 5 mg, 0.25 to 3 mg, and 1 to 2 mg.

27. The pellet of claim 1 which is formulated for administration 0 to 2 hours or 30 to 60 minutes before the subject of treatment goes to bed.

28. A method of treating insomnia, comprising administering to a subject the pellet of claim 1.

29-30. (canceled)

31. A process for preparing a pellet, comprising:
a) making a core solution comprising melatonin, a coating layer solution comprising a polymer, and an IR layer solution comprising melatonin;
b) applying the core solution to a pellet and then allowing the pellet to dry until the pellet reaches a temperature of no less than 45° C.;
c) applying the coating layer solution to the pellet followed by spraying the pellet with a rinsing solution;
d) optionally curing the pellet for 2 to 6 hours or 3 to 5 hours; and
e) applying the IR layer solution to the pellet and then allowing the pellet to dry.
